(19) United States
(12) Patent Application Publication
(10) Pub. No.: US 2023/0031534 A1
(43) Pub. Date: Feb. 2, 2023

(54) METHODS FOR ALLEVIATING ENTERIC PATHOGENIC BACTERIUM-ASSOCIATED DISORDER AND INHIBITING GROWTH OF ENTERIC PATHOGENIC BACTERIA

(71) Applicant: GLAC BIOTECH CO., LTD., Tainan City (TW)

(72) Inventors: Hsieh-Hsun HO, Tainan City (TW); Yi-Wei Kuo, Tainan City (TW); Ching-Wei Chen, Tainan City (TW); Jui-Fen Chen, Tainan City (TW); Shu-Hui Chen, Tainan City (TW)

(21) Appl. No.: 17/573,162

(22) Filed: Jan. 11, 2022

(30) Foreign Application Priority Data
Jul. 26, 2021 (CN) 202110848624.9

(51) Int. Cl. A61K 35/747; A61P 31/04; C12N 1/20; A23L 33/135; C12R 1/225

(52) U.S. Cl. CPC A61K 35/747; A61P 31/04; C12N 1/205; A23L 33/135; C12R 2001/225

(57) ABSTRACT

Disclosed herein are methods for alleviating an enteric pathogenic bacterium-associated disorder and inhibiting growth of enteric pathogenic bacteria using a composition containing cultures of Lactobacillus rhamnosus MP108 which is deposited at the China General Microbiological Culture Collection Center (CGMCC) under an accession number CGMCC 21225, and Lactobacillus paracasei MP137 which is deposited at the CGMCC under an accession number CGMCC 21224.

### CROSS-REFERENCE TO RELATED APPLICATION

This application claims priority of Chinese Invention Patent Application No. 202110848624.9, filed on Jul. 26, 2021.

### FIELD

The present disclosure relates to methods for alleviating an enteric pathogenic bacterium-associated disorder and inhibiting growth of enteric pathogenic bacteria using a composition containing cultures of two lactic acid bacterial strains.

### BACKGROUND

Enteric pathogenic bacteria generally exist in the intestines of humans and animals, water, and spoiled food. Common enteric pathogenic bacteria include Salmonella enterica, Listeria monocytogenes, Escherichia coli, and Vibrio parahaemolyticus. Enteric pathogenic bacterium-associated disorders include diarrhea, gastroenteritis (such as gastritis and enteritis), ulcerative colitis (UC), typhoid fever, bacteremia, etc.

Enteric pathogenic bacterium-associated disorders are typically treated with antibiotics. However, these antibiotics might cause enteric pathogens to develop antibiotic resistance, and might cause severe side effects and adverse effects.

Probiotics are resident normal flora of the intestinal tract and believed to play important roles in regulating proper intestinal immunity and digestion by balancing intestinal microflora. These beneficial microorganisms are widely used as live microbial dietary supplements and can help restoring intestinal microfloral balance. Many species of lactic acid bacteria (LAB) are conferred with the generally recognized as safe (GRAS) status, and are widely used as probiotics. Common LAB include Lactobacillus spp., Lactococcus spp., Pediococcus spp., Streptococcus spp., Enterococcus spp., Bifidobacterium spp., Bacillus spp., Leuconostoc spp., etc.

Previous studies have demonstrated that certain strains of LAB are effective against enteric pathogens infection. For example, CM 102604854 B discloses that Lactobacillus rhamnosus MP108 and Lactobacillus paracasei MP137 have been demonstrated to have ability to inhibit the adsorption of Salmonella enterica subsp. enterica (BCRC 10744) and Escherichia coli (BCRC 15372) to the gastrointestinal tract or ability to replace Salmonella enterica subsp. enterica and Escherichia coli to adsorb to the gastrointestinal tract, hence being capable of achieving the effect of anti-enteric pathogen infection.

In spite of the aforesaid, there is still a need to develop a new strategy that can be utilized for alleviating an enteric pathogenic bacterium-associated disorder and inhibiting growth of enteric pathogenic bacteria.

### SUMMARY

Therefore, in a first aspect, the present disclosure provides a method for alleviating an enteric pathogenic bacterium-associated disorder which can alleviate at least one of the drawbacks of the prior art.

The method includes administering to a subject in need thereof a composition containing cultures of Lactobacillus rhamnosus MP108 which is deposited at the China General Microbiological Culture Collection Center (CGMCC) under an accession number CGMCC 21225, and Lactobacillus paracasei MP137 which is deposited at the CGMCC under an accession number CGMCC 21224.

In a second aspect, the present disclosure provides a method for inhibiting growth of enteric pathogenic bacteria, which can alleviate at least one of the drawbacks of the prior art, and which includes applying the aforesaid composition onto an object.

### DETAILED DESCRIPTION

It is to be understood that, if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to”, and that the word “comprises” has a corresponding meaning.

Unless otherwise defined, all technical and scientific terms used herein have the meaning commonly understood by a person skilled in the art to which this disclosure belongs. One skilled in the art will recognize many methods and materials similar or equivalent to those described herein, which could be used in the practice of this disclosure. Indeed, this disclosure is in no way limited to the methods and materials described.

The present disclosure provides a method for alleviating an enteric pathogenic bacterium-associated disorder, which includes administering to a subject in need thereof a composition containing cultures of Lactobacillus rhamnosus MP108 which is deposited at the China General Microbiological Culture Collection Center (CGMCC) under an accession number CGMCC 21225, and Lactobacillus paracasei MP137 which is deposited at the CGMCC under an accession number CGMCC 21224.

As used herein, the term “alleviating” or “alleviation” refers to at least partially reducing, ameliorating, relieving, controlling, treating or eliminating one or more clinical signs of a disease or disorder; and lowering, delaying, stopping or reversing the progression of severity regarding the condition or symptom being treated and preventing or decreasing the likelihood or probability thereof.

As used herein, the term “administering” or “administration” means introducing, providing or delivering the abovementioned composition to a subject showing condition(s) or symptom(s) of an enteric pathogenic bacterium-related disorder by any suitable routes to perform its intended function.

As used herein, the term “subject” refers to any animal of interest, such as humans, monkeys, cows, sheep, horses, pigs, goats, dogs, cats, mice, and rats. In certain embodiments, the subject is a human.

According to the present disclosure, the enteric pathogenic bacterium-associated disorder may be caused by an enteric pathogenic bacterium selected from the group consisting of Salmonella enterica subsp. enterica, Listeria monocytogenes, Escherichia coli, Vibrio parahaemolyticus, Staphylococcus aureus, Shigella boydii, Shigella dysenteriae, Klebsiella pneumoniae, Yersinia enterocolitica, Proteus vulgaris, and combinations thereof.

According to the present disclosure, the culture of each lactic acid bacterial strain may be prepared by culturing the abovementioned lactic acid bacterial strain in a liquid or solid medium suitable for growth and/or proliferation thereof.

In certain embodiments, the culture of each lactic acid bacterial strain may be prepared by culturing the abovementioned lactic acid bacterial strain in an amount of 10⁶ CFU/mL, to 10¹⁰ CFU/mL in a liquid or solid medium suitable for growth thereof. In an exemplary embodiment, the abovementioned lactic acid bacterial strain is cultivated in an amount of 10⁷ CFU/mL to 10⁹ CFU/mL in a liquid or solid medium suitable for growth thereof.

As used herein, the term “culturing” can be used interchangeably with other terms such as “fermentation” and “cultivation”.

The procedures and conditions for culturing the lactic acid bacterial strain may be adjusted according to practical requirements. In this regard, those skilled in the art may refer to journal articles, e.g., Hsieh, P. S. et al. (2013), New Microbiol., 36:167-179.

According to the present disclosure, the liquid medium suitable for culturing the lactic acid bacterial strain may include, but is not limited to, MRS (De Man, Rogosa and Sharpe) broth and MRS broth containing cysteine.

In certain embodiments, the culture of each lactic acid bacterial strain is a liquid culture.

In certain embodiments, the liquid culture may have a total bacterial concentration ranging from 10⁶ CFU/mL to 10¹⁰ CFU/mL (a mixture of the liquid cultures of the aforesaid two lactic acid bacterial strains may also have such total bacterial concentration). In an exemplary embodiment, the liquid culture has a total bacterial concentration ranging from 10⁷ CFU/mL to 10⁹ CFU/mL (a mixture of the liquid cultures of the aforesaid two lactic acid bacterial strains may also have such total bacterial concentration).

In certain embodiments, a number ratio of Lactobacillus rhamnosus MP108 and Lactobacillus paracasei MP137 in the composition ranges from 1:0.43 to 1:2.33. In an exemplary embodiment, the number ratio of Lactobacillus rhamnosus MP108 and Lactobacillus paracasei MP137 in the composition is 1:1.

According to the present disclosure, the liquid culture may be heat-inactivated.

According to the present disclosure, the heat inactivation of the liquid culture may be conducted at 100° C. for 30 minutes to 1 hour. In an exemplary embodiment, the heat inactivation of the liquid culture is conducted at 100° C. for 1 hour.

According to the present disclosure, the liquid culture may be substantially free of cells.

In certain embodiments, when the liquid culture is substantially free of cells, the volume ratio of the liquid cultures of Lactobacillus rhamnosus MP108 and Lactobacillus paracasei MP137 in the composition ranges from 1:0.43 to 1:2.33. In an exemplary embodiment, the volume ratio of the liquid cultures of Lactobacillus rhamnosus MP108 and Lactobacillus paracasei MP137 in the composition is 1:1.

As used herein, the term “substantially free of” means that the liquid culture lacks a significant amount of a specified component (i.e., lactic acid bacterial cells). In certain embodiments, the amount of the lactic acid bacterial cells does not have a measurable effect on the properties of the liquid culture. In other embodiments, the liquid culture is completely free of the bacterial cells.

According to the present disclosure, the liquid culture which is substantially free of cells is obtained by subjecting a culture formed after culturing the lactic acid bacterial strain to a separation treatment to remove bacterial cells therefrom.

According to the present disclosure, the separation treatment may be performed using techniques well-known to those skilled in the art. Examples of the separation treatment may include, but are not limited to, filtration, centrifugation (such as multi-stage centrifugation), concentration, and combinations thereof.

In an exemplary embodiment, the liquid culture which is substantially free of cells is obtained by subjecting the culture formed after culturing the lactic acid bacterial strain to a centrifugation treatment.

According to the present disclosure, the enteric pathogenic bacterium-associated disorder may be selected from the group consisting of diarrhea (such as infectious diarrhea and traveler's diarrhea), gastroenteritis (such as gastritis and enteritis), ulcerative colitis (UC), typhoid fever, bacteremia, necrotizing enterocolitis (NEC), dysentery, and combinations thereof.

According to the present disclosure, the composition may be formulated as a food product using a standard technique well known to one of ordinary skill in the art. For example, the composition may be directly added to an edible material or may be used to prepare an intermediate composition (e.g., a food additive or a premix) suitable to be subsequently added to the edible material.

As used herein, the term “food product” refers to any article or substance that can be ingested by a subject into the body thereof. Examples of the food product may include, but are not limited to, milk powders, fermented milk, yogurt, butter, beverages (e.g., tea, coffee, etc.), functional beverages, a flour product, baked foods, confectionery, candies, fermented foods, animal feeds, health foods, infant foods, and dietary supplements.

According to the present disclosure, the composition may be prepared in the form of a pharmaceutical composition. The pharmaceutical composition may be formulated into a suitable dosage form for oral, parenteral or topical administration using technology well known to those skilled in the art.

According to the present disclosure, the suitable dosage form for oral administration includes, but is not limited to, sterile powders, tablets, troches, lozenges, sustained film-coated tablets, oral ointments, pellets, capsules, dispersible powders or granules, solutions, suspensions, emulsions, syrup, elixir, slurry, drops, and the like.

For parenteral administration, the pharmaceutical composition according to the present disclosure may be formulated into an injection, e.g., a sterile aqueous solution or a dispersion.

The pharmaceutical composition according to the present disclosure may be administered via one of the following parenteral routes: intraperitoneal injection, intrapleural injection, intramuscular injection, intravenous injection, intraarterial injection, intraarticular injection, intrasynovial injection, intrathecal injection, intracranial injection, intraepidermal injection, subcutaneous injection, intradermal injection, intralesional injection, and sublingual administration. In certain embodiments, the pharmaceutical composition may be administered via intralesional injection.

According to the present disclosure, the pharmaceutical composition may be formulated into an external preparation suitable for topical application to the skin using technology well known to those skilled in the art. The external preparation includes, but is not limited to, emulsions, gels, ointments, creams, patches, liniments, powder, aerosols, sprays, lotions, serums, pastes, foams, drops, suspensions, salves, and bandages.

The pharmaceutical composition according to the present disclosure may further include a pharmaceutically acceptable carrier widely employed in the art of drug-manufacturing. For instance, the pharmaceutically acceptable carrier may include one or more of the following agents: solvents, buffers, emulsifiers, suspending agents, decomposers, disintegrating agents, dispersing agents, binding agents, excipients, stabilizing agents, chelating agents, diluents, gelling agents, preservatives, fillers, wetting agents, lubricants, absorption delaying agents, liposomes, and the like. The choice and amount of the aforesaid agents are within the expertise and routine skills of those skilled in the art.

The dose and frequency of administration of the composition according to the present disclosure may vary depending on the following factors: the severity of the illness or disorder to be treated, routes of administration, and age, physical condition and response of the subject to be treated. In general, the composition may be administered in a single dose or in several doses.

The present disclosure also provides a method for inhibiting growth of enteric pathogenic bacteria, which includes applying the aforesaid composition onto an object. The enteric pathogenic bacteria may be the same as the aforesaid enteric pathogenic bacterium causing the enteric pathogenic bacterium-associated disorder.

According to the present disclosure, the object may be a medical device, a medical instrument, a food preparation countertop, a food packaging countertop, a manufacturing countertop, a consumer good, a water treatment system, or a water delivery system.

In certain embodiments, the object may be selected from the group consisting of a denture, a mouth guard, a dairy line, a water line, an adhesive bandage, a component of a water treatment facility, a medical instrument, a dental instrument, food industry processing instruments, hospital tables and beds, an animal water dish, a washing machine, a dish washer, a towel, a dish, a bowl, a utensil, a cup, a glass, a cutting board, a dish drying tray, a sink, a restroom, a toilet seat, a line used in food and beverage manufacturing, an animal water dish, a food storage container, a beverage storage container, a plate, a fork, a knife, and a spoon.

The present disclosure will be further described by way of the following examples. However, it should be understood that the following examples are intended solely for the purpose of illustration and should not be construed as limiting the present disclosure in practice.

### EXAMPLES

### General Experimental Materials:

### 1. Lactic Acid Bacterial (LAB) Strains

Lactobacillus rhamnosus MP108 and Lactobacillus paracasei MP137 (which are disclosed in CN 102604854 B) have been deposited at the Deutsche Sammlung von Mikroorganismen and Zellkulturen (DSMZ) GmbH (Inhoffenstraβe 7B, 38124 Braunschweig, Germany), and have also been deposited at the China General Microbiological Culture Collection Center (CGMCC) of Chinese Academy of Sciences, the Institute of Microbiology (No. 1, West Beichen Rd., Chaoyang District, Beijing 100101, China) in accordance with the Budapest Treaty.

The relevant information regarding each of the LAB strains (including accession number and date of deposit) is listed in Table 1 below.

TABLE 1

| LAB strains | Accession number | Date of deposit |
|---|---|---|
| Lactobacillus | DSM 24229 | Nov. 19, 2010 |
| rhamnosus MP108 | CGMCC 21225 | Nov. 23, 2020 |
| Lactobacillus | DSM 24230 | Nov. 10, 2010 |
| paracasei MP137 | CGMCC 21224 | Nov. 23, 2020 |

In addition, the applicant isolated the following LAB strains for them to serve as comparative LAB strains:

(1) Lactobacillus rhamnosus L-68

Lactobacillus rhamnosus L-68 used in the following experiments was isolated from the feces of a healthy subject by the applicant using BD Difco™ Lactobacilli MRS Agar. Preliminary morphological characterization test showed that Lactobacillus rhamnosus L-68 is heterofermentative facultative anaerobic, Gram-positive, non-motile, catalase negative, and non-spore-forming, and exhibits optimal growth at 37° C.±1° C. It was found that when Lactobacillus rhamnosus L-68 performs glucose metabolism, no gas is generated. The cells of Lactobacillus rhamnosus L-68 are rod-shaped with oval ends, and occur singly or in short chains. Identification of Lactobacillus rhamnosus L-68 was verified using analytical profile index (API) test and 16S ribosomal DNA analysis according to procedures known to those skilled in the art (data not shown).

(2) Lactobacillus paracasei L-30

Lactobacillus paracasei L-30 used in the following experiments was isolated from the feces of a healthy subject by the applicant using BD Difco™ Lactobacilli MRS Agar. Preliminary morphological characterization test showed that Lactobacillus paracasei L-30 is heterofermentative facultative anaerobic, Gram-positive, non-motile, catalase negative, and non-spore-forming, and exhibits optimal growth at 37° C.±1° C. It was found that when Lactobacillus paracasei L-30 performs glucose metabolism, no gas is generated. The cells of Lactobacillus paracasei L-30 are rod-shaped with oval ends, and occur in short chains. Identification of Lactobacillus paracasei L-30 was verified using analytical profile index (API) test and 16S ribosomal DNA analysis according to procedures known to those skilled in the art (data not shown).

### 2.Preparation of Bacterial Suspension of LAB Strain

A respective one of the four LAB strains described in section 1 of “General Experimental Materials” was inoculated in a MRS (De Man, Rogosa and Sharpe) broth (Cat. No. 288130, BD Difco) supplemented with 0.05% (w/w) cysteine, followed by cultivation in an incubator (37° C., 5% CO₂) for 24 hours to obtain a respective inoculum. Thereafter, the respective inoculum was inoculated in an amount of 2% (v/v) into a MRS broth, followed by subculturing in an incubator (37° C., 5% CO) for 24 hours. The resultant cell culture was collected, followed by adding a suitable amount of a MRS broth, so as to obtain a bacterial suspension having a bacterial concentration of 1×10⁹ CFU/mL.

### 3. Enteric Pathogenic Bacterial Strains

Four enteric pathogenic bacterial strains used in the following experiments were purchased from the Bioresource Collection and Research Center (BCRC) of the Food Industry Research and Development Institute (FIRDI) (No. 331, Shih-Pin Rd., Hsinchu City 300, Taiwan). The relevant information regarding each of the enteric pathogenic bacterial strains is listed in Table 2 below.

TABLE 2

| Enteric pathogenic | |
|---|---|
| bacterial strains | Accession number |
| Salmonella enterica | BCRC 10747 |
| subsp. enterica | |
| Listeria monocytogenes | BCRC 14845 |
| Escherichia coli | BCRC 11634 |
| Vibrio parahaemolyticus | BCRC 12974 |

### 4. Preparation of Bacterial Suspension of Enteric Pathogenic Bacterial Strain

A respective one of the four enteric pathogenic bacterial strains described in section 3 of “General Experimental Materials” was cultivated using the corresponding medium and cultivation conditions shown in Table 3 in an incubator for 20 hours to obtain a respective inoculum. Thereafter, the respective inoculum was subcultured in an amount of 2% (v/v) using the corresponding medium and cultivation conditions shown in Table 3 in an incubator, so as to obtain a bacterial suspension having a bacterial concentration ranging from 1×10⁷ CFU/mL to 9×10⁷ CFU/mL.

TABLE 3

| Enteric pathogenic | | Cultivation |
|---|---|---|
| bacterial strains | Medium | conditions |
| Salmonella enterica | Nutrient broth | 37 ° C., 5% CO₂ |
| subsp. enterica | (NB) (BD Bioscience, | |
| | Cat. No. 234000) | |
| Escherichia coli | Nutrient broth | 37° C., 5% CO₂ |
| | (as mentioned above) | |
| Listeria | Brain heart infusion | 37° C., 5% CO₂ |
| monocytogenes | (BHI) medium | |
| | (BD Bioscience, Cat. | |
| | No. R452472) | |
| Vibrio | Tryptic soy broth | 25° C., 5% CO₂ |
| parahaemolyticus | (TSB) (BD Bioscience, | |
| | Cat. No. R112996) | |
| | supplemented with | |
| | 2.5% NaCl (Sigma, | |
| | Cat. No. S5886) | |

### General Procedures:

### 1. Data Analysis

All the experiments described below were performed in triplicate. The experimental data are expressed as mean.

### Example 1. Evaluation for the Effect of Liquid Culture of LAB Strain According to this Disclosure Against Enteric Pathogenic Bacterial Strain

### Materials and Methods:

### A. Preparation of MRS Agar Plate

The MRS agar plate used in the following experiments was prepared by adding 1.5% (w/v) of agar powder to a MRS broth and using technology well-known to those skilled in the art.

### B. Preparation of Top Agar Medium

A respective one of the three mediums described in Table 3 was added with 1.5% (w/v) of agar powder, followed by sterilization at 121° C. for 15 minutes. The respective resultant melted agar medium (serving as a top agar medium) was then placed in a 45° C. water bath for subsequent use.

### C. Determination of Anti-Pathogenic Efficacy

The anti-pathogenic efficacy of the liquid culture of each LAB stain was determined using a double agar overlay method slightly modified from that described by Chen, Y. T. et al. (2020), Lett. Appl. Microbio., 70(4):310-317. Briefly, each of the four bacterial suspensions prepared in section 2 of “General Experimental Materials” (in a suitable amount) served as a single LAB group (i.e., a corresponding one of comparative groups S1 to S4), as shown in Table 4 below.

TABLE 4

| Group | LAB strain applied |
|---|---|
| Comparative group S1 | Lactobacillus rhamnosus MP108 |
| Comparative group S2 | Lactobacillus paracasei MP137 |
| Comparative group S3 | Lactobacillus rhamnosus L-68 |
| Comparative group S4 | Lactobacillus paracasei L-30 |

In addition, the bacterial suspensions of Lactobacillus rhamnosus MP108 and Lactobacillus paracasei MP137 prepared in section 2 of “General Experimental Materials” (in suitable amounts) were mixed in different volume ratios to obtain 5 suspension mixtures (i.e., suspension mixtures 1 to 5). Each of the five suspension mixtures served as a mixture LAB group (i.e., a corresponding one of experimental groups M1 to M5), as shown in Table 5 below. Each group had a total bacterial concentration of 1×10⁹ CFU/mL.

TABLE 5

| | | Volume ratio of Lactobacillus |
|---|---|---|
| | Suspension | rhamnosus MP108 and |
| Group | mixture | Lactobacillus paracasei MP137 |
| Experimental | 1 | 1:0.11 |
| group M1 | | |
| Experimental | 2 | 1:0.43 |
| group M2 | | |
| Experimental | 3 | 1:1 |
| group M3 | | |
| Experimental | 4 | 1:2.33 |
| group M4 | | |
| Experimental | 5 | 1:9 |
| group M5 | | |

Thereafter, about 1 mL of the bacterial suspension of the respective comparative group or the suspension mixture of the respective experimental group was collected using a sterilized cotton swab by dipping, and then a line with a width of approximately 2 cm was formed along the diameter of the MRS agar plate prepared in section A of this example using the cotton swab with the collected suspension or suspension mixture, followed by cultivation in an incubator (37° C.) for 48 hours, so that the respective group formed a growth zone with a width of approximately 2 cm on the surface of the MRS agar medium. In addition, a MRS broth being free from any LAB strain (serving as a blank control group) was subjected to the same procedure.

Next, the melted NB agar medium (i.e., the top agar medium) prepared in section B of this example was poured into the MRS agar plate of each group to evenly overlay the MRS agar medium. After the NB agar medium had solidified to form a double-layer agar (DLA) medium, the bacterial suspension of Salmonella enterica subsp. enterica prepared in section 4 of “General Experimental Materials” was collected using a sterilized cotton swab by dipping, and was then evenly coated onto the surface of the DLA medium (i.e., the top agar medium) of each group, followed by cultivation in an incubator (37° C., 5% CO₂) for 24 hours. The effect of each group in inhibiting the growth of Salmonella enterica subsp. enterica was evaluated by measuring the width of the inhibition zone formed by each group on the surface of the DLA medium.

The inhibitory effects of each group on Escherichia coli, Listeria monocytogenes, and Vibrio parahaemolyticus were determined according to the procedure described above, except that the bacterial suspension of Escherichia coli was coated onto the surface of the DLA medium containing the NB agar medium as a top agar medium, the bacterial suspension of Listeria monocytogenes was coated onto the surface of the DLA medium containing the BHI agar medium as a top agar medium, or the bacterial suspension of Vibrio parahaemolyticus was coated onto the surface of the DLA medium containing the TSB agar medium supplemented with 2.5% NaCl as a top agar medium.

The data thus obtained were analyzed according to section 1 of “General Procedures”.

### Results:

Table 6 shows the width of the inhibition zone determined in each group. As shown in Table 6, for each enteric pathogenic bacterial strain, the width of the inhibition zone determined in each of the experimental groups M1 to M5 was significantly larger than those determined in the comparative groups S3 to S4. Moreover, the width of the inhibition zone determined in each of the experimental groups M2 to M4 was larger than those determined in the comparative groups S1 to S2.

The aforesaid result suggests that Lactobacillus rhamnosus MP108 and Lactobacillus paracasei MP137, when used in combination to prepare a suspension mixture, can synergistically exhibit satisfactory efficacy in inhibiting growth of enteric pathogenic bacteria.

TABLE 6

| | Width of inhibition zone (cm) | | | |
|---|---|---|---|---|
| | Salmonella enterica | Listeria | Escherichia | Vibrio |
| Group | subsp. enterica | monocytogenes | coli | parahaemolyticus |
| Blank control | 0.0 | 0.0 | 0.0 | 0.0 |
| group | | | | |
| Comparative | 3.5 | 3.2 | 4.1 | 3.5 |
| group S1 | | | | |
| Comparative | 3.7 | 3.5 | 3.4 | 4.0 |
| group S2 | | | | |
| Comparative | 2.3 | 2.6 | 3.1 | 2.1 |
| group S3 | | | | |
| Comparative | 2.6 | 2.1 | 2.8 | 2.2 |
| group S4 | | | | |
| Experimental | 3.4 | 3.7 | 3.2 | 4.2 |
| group M1 | | | | |
| Experimental | 3.9 | 4.2 | 4.2 | 4.3 |
| group M2 | | | | |
| Experimental | 4.7 | 4.6 | 5.1 | 4.9 |
| group M3 | | | | |
| Experimental | 4.1 | 3.9 | 4.6 | 4.3 |
| group M4 | | | | |
| Experimental | 3.6 | 3.3 | 3.9 | 4.1 |
| group M5 | | | | |

### Example 2. Evaluation for the Effect of Cell Culture Supernatant of LAB Strain According to this Disclosure Against Enteric Pathogenic Bacterial Strain

### Materials and Methods:

A. Preparation of Cell Culture Supernatant of LAB Strain A respective one of the four bacterial suspensions prepared in section 2 of “General Experimental Materials” was subjected to centrifugation at 4,000 rpm and 4° C. for 10 minutes. The resultant cell culture supernatant was collected for subsequent use.
B. Co-Cultivation of Enteric Pathogenic Bacterial Strain with Cell Culture Supernatant of LAB Strain

Each of the four cell culture supernatants prepared in section A of this example (in a suitable amount) served as a single LAB group (i.e., a corresponding one of comparative groups S1 to S4), as shown in Table 7 below.

TABLE 7

| Group | LAB strain applied |
|---|---|
| Comparative group S1 | Lactobacillus rhamnosus MP108 |
| Comparative group S2 | Lactobacillus paracasei MP137 |
| Comparative group S3 | Lactobacillus rhamnosus L-68 |
| Comparative group S4 | Lactobacillus paracasei L-30 |

In addition, the cell culture supernatants of

Lactobacillus rhamnosus MP108 and Lactobacillus paracasei MP137 prepared in section A of this example (in suitable amounts) were mixed in different volume ratios to obtain 5 supernatant mixtures (i.e., supernatant mixtures 1 to 5). Each of the five supernatant mixtures served as a mixture LAB group (i.e., a corresponding one of experimental groups M1 to M5), as shown in Table 8 below.

TABLE 8

| | | Volume ratio of Lactobacillus |
|---|---|---|
| | Supernatant | rhamnosus MP108 and |
| Group | mixture | Lactobacillus paracasei MP137 |
| Experimental | 1 | 1:0.11 |
| group M1 | | |
| Experimental | 2 | 1:0.43 |
| group M2 | | |
| Experimental | 3 | 1:1 |
| group M3 | | |
| Experimental | 4 | 1:2.33 |
| group M4 | | |
| Experimental | 5 | 1:9 |
| group M5 | | |

100 μL of the cell culture supernatant of the respective comparative group or the supernatant mixture of the respective experimental group was added to 4.8 mL of a nutrient broth to from a liquid mixture. In addition, 200 μL of a MRS broth being free from any LAB strain (serving as a blank control group) was added to 4.8 mL of a nutrient broth to from a liquid mixture.

Thereafter, 100 μL of the bacterial suspension of Salmonella enterica subsp. enterica prepared in section 4 of “General Experimental Materials” (which had a bacterial concentration of 1×10⁷ CFU/mL) was added to the liquid mixture of the respective group, followed by cultivation in an incubator (37° C., 5% CO₂) for 20 hours to obtain a co-cultivation product.

In addition, the procedures for preparing the co-cultivation products of Escherichia coli, Listeria monocytogenes, and Vibrio parahaemolyticus for each group were similar to that of Salmonella enterica subsp. enterica, except that the corresponding medium and cultivation conditions shown in Table 3 were used, the cultivation time for Listeria monocytogenes and Vibrio parahaemolyticus was 24 hours, and the cultivation time for Escherichia coli was 48 hours.

### C. Determination of Inhibition Rate

According to the type of enteric pathogenic bacterial strain used for co-cultivation, a respective one of the co-cultivation products prepared in section B of this example was subjected to 10-fold serial dilution with the corresponding medium shown in Table 3, so as to obtain ten dilutions (prepared using dilution factors of 10¹, 10², 10³, 10⁴, 10⁵, 10⁶, 10⁷, 10⁸, 10⁹, and 10¹⁰. 100 μL of a respective one of the ten dilutions was evenly coated onto a corresponding agar medium as described in section C of Example 1 using spread plate technique, followed by cultivation in an incubator (37° C., 5% CO₂) for 24 hours to 48 hours.

The agar medium formed by a suitable dilution factor was selected for bacterial colony counting. The number of colonies of enteric pathogenic bacterial strain on the agar medium of each group was counted, and the inhibition rate (I) was further calculated using the following Equation (I):

A=[(B−C)/B]×100 (I)

where A=inhibition rate (%)
B=number of colonies of enteric pathogenic bacterial strain in blank control group
C=number of colonies of enteric pathogenic bacterial strain in respective group

The data thus obtained were analyzed according section 1 of “General Procedures”.

### Results:

Table 9 shows the inhibition rate of each group. As shown in Table 9, for each enteric pathogenic bacterial strain, the inhibition rate determined in each of the experimental groups M1 to M5 was significantly higher than those determined in the comparative groups S3 to S4. Moreover, the inhibition rate determined in each of the experimental groups M2 to M4 was higher than those determined in the comparative groups S1 to S2.

TABLE 9

| | Inhibition rate (%) | | | |
|---|---|---|---|---|
| | Salmonella enterica | Listeria | Escherichia | Vibrio |
| Group | subsp. enterica | monocytogenes | coli | parahaemolyticus |
| Blank control | 0.00 | 0.00 | 0.00 | 0.00 |
| group | | | | |
| Comparative | 76.23 | 71.23 | 76.51 | 62.32 |
| group S1 | | | | |
| Comparative | 71.23 | 56.87 | 68.46 | 66.38 |
| group S2 | | | | |
| Comparative | 51.23 | 49.85 | 41.23 | 46.52 |
| group S3 | | | | |
| Comparative | 50.23 | 49.81 | 39.56 | 42.09 |
| group S4 | | | | |
| Experimental | 75.51 | 62.34 | 71.61 | 72.34 |
| group M1 | | | | |
| Experimental | 76.35 | 87.43 | 83.24 | 73.55 |
| group M2 | | | | |
| Experimental | 93.46 | 94.22 | 92.34 | 89.46 |
| group M3 | | | | |
| Experimental | 87.11 | 86.52 | 84.52 | 72.13 |
| group M4 | | | | |
| Experimental | 77.56 | 63.87 | 75.34 | 65.34 |
| group M5 | | | | |

The aforesaid result suggests that Lactobacillus rhamnosus MP108 and Lactobacillus paracasei MP137, when used in combination to prepare a supernatant mixture, can synergistically exhibit satisfactory efficacy in inhibiting growth of enteric pathogenic bacteria.

Summarizing the above test results, it is clear that the abovementioned bacterial suspension mixture or supernatant mixture of Lactobacillus rhamnosus MP108 and Lactobacillus paracasei MP137 can act effectively against enteric pathogenic bacterial infection, and hence can alleviate an enteric pathogenic bacterium-associated disorder.

In the description above, for the purposes of explanation, numerous specific details have been set forth in order to provide a thorough understanding of the embodiments. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A method for alleviating an enteric pathogenic bacterium-associated disorder, comprising administering to a subject in need thereof a composition containing cultures of Lactobacillus rhamnosus MP108 which is deposited at the China General Microbiological Culture Collection Center (CGMCC) under an accession number CGMCC 21225, and Lactobacillus paracasei MP137 which is deposited at the CGMCC under an accession number CGMCC 21224.

2. The method as claimed in claim 1, wherein each of the cultures is a liquid culture.

3. The method as claimed in claim 2, wherein a number ratio of Lactobacillus rhamnosus MP108 and Lactobacillus paracasei MP137 in the composition ranges from 1:0.43 to 1:2.33.

4. The method as claimed in claim 3, wherein the liquid culture is heat-inactivated.

5. The method as claimed in claim 2, wherein the liquid culture is substantially free of cells.

6. The method as claimed in claim 5, wherein a volume ratio of the liquid cultures of Lactobacillus rhamnosus MP108 and Lactobacillus paracasei MP137 in the composition ranges from 1:0.43 to 1:2.33.

7. The method as claimed in claim 1, wherein the enteric pathogenic bacterium-associated disorder is caused by an enteric pathogenic bacterium selected from the group consisting of Salmonella enterica subsp. enterica, Listeria monocytogenes, Escherichia coli, Vibrio parahaemolyticus, Staphylococcus aureus, Shigella boydii, Shigella dysenteriae, Klebsiella pneumoniae, Yersinia enterocolitica, Proteus vulgaris, and combinations thereof.

8. The method as claimed in claim 1, wherein the composition is formulated as a food product.

9. The method as claimed in claim 1, wherein the composition is formulated as a pharmaceutical composition.

10. The method as claimed in claim 9, wherein the pharmaceutical composition is in a dosage form selected from the group consisting of an oral dosage form, a parenteral dosage form, and a topical dosage form.

11. A method for inhibiting growth of enteric pathogenic bacteria, comprising applying a composition onto an object, wherein the composition contains cultures of Lactobacillus rhamnosus MP108 which is deposited at the China General Microbiological Culture Collection Center (CGMCC) under an accession number CGMCC 21225, and Lactobacillus paracasei MP137 which is deposited at the CGMCC under an accession number CGMCC 21224.

12. The method as claimed in claim 11, wherein each of the cultures is a liquid culture.

13. The method as claimed in claim 12, wherein a number ratio of Lactobacillus rhamnosus MP108 and Lactobacillus paracasei MP137 in the composition ranges from 1:0.43 to 1:2.33.

14. The method as claimed in claim 13, wherein the liquid culture is heat-inactivated.

15. The method as claimed in claim 12, wherein the liquid culture is substantially free of cells.

16. The method as claimed in claim 15, wherein a volume ratio of the liquid cultures of Lactobacillus rhamnosus MP108 and Lactobacillus paracasei MP137 in the composition ranges from 1:0.43 to 1:2.33.

17. The method as claimed in claim 11, wherein the enteric pathogenic bacteria are selected from the group consisting of Salmonella enterica subsp. enterica, Listeria monocytogenes, Escherichia coli, Vibrio parahaemolyticus, Staphylococcus aureus, Shigella boydii, Shigella dysenteriae, Klebsiella pneumoniae, Yersinia enterocolitica, Proteus vulgaris, and combinations thereof.
